(19) United States
(12) Patent Application Publication
(10) Pub. No.: US 2024/0062856 A1
(43) Pub. Date: Feb. 22, 2024

(54) MEDICAL SURVEY TRIGGER AND PRESENTATION

(71) Applicant: Medtronic, Inc., Minneapolis, MN (US)

(72) Inventors: Tarek D. Haddad, Minneapolis, MN (US); Lawrence C. Johnson, San Francisco, CA (US); Chris K. Reedy, Denver, CO (US); Joey J. Hendrickson, Chisago City, MN (US); Manish K. Singh, Plymouth, MN (US); Kevin Joseph Pochatila, Minneapolis, MN (US); Nirav A. Patel, Dublin, OH (US); Linda Z. Massie, Lake Mary, FL (US); Noreli C. Franco, Fridley, MN (US); Michael Erich Jordan, St. Paul, MN (US); Adam V. Dewing, Roseville, MN (US); Vamshi Poornima Yerrapragada Durga, Circle Pines, MN (US); Katy A. Muckala, Arden Hills, MN (US); Sairaghunath B. Godithi, Medina, MN (US); Jan Audrey Loleng San Diego, Minneapolis, MN (US); Hannah Rose Griebel, Manchester (GB); Vivian Wing See To, Minneapolis, MN (US); Evan J. Stanelle, St. Paul, MN (US); Rahul Kanwar, Rochester, MN (US); Dana M. Soderlund, Plymouth, MN (US)

(21) Appl. No.: 18/261,019

(22) PCT Filed: Feb. 7, 2022
(86) PCT No.: PCT/US2022/015399

§ 371 Date: Jul. 11, 2023

Related U.S. Application Data
(60) Provisional application No. 63/147,581, filed on Feb. 9, 2021.

(51) Int. Cl. G16H 10/20; A61B 5/361; A61B 5/00

(52) U.S. Cl. CPC G16H 10/20; A61B 5/361; A61B 5/0006; A61B 5/0022; A61B 5/686; A61B 5/74; A61B 2560/0468; A61B 2560/0487

(57) ABSTRACT

A method for processing patient data includes prompting a patient to complete a survey based on one or more of data received from an implantable medical device, a first time from an enrollment in a study related to the implantable medical device, a second time since a last survey, a medical event, or a detection of the patient in a geofenced area. The method may further include receiving input from the patient in response to the survey, and sending the input from the patient to a database.

### FIELD

This disclosure generally relates to systems including medical devices and, more particularly, to monitoring of patient health using such systems.

### BACKGROUND

A variety of devices are configured to configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect health events, such as episodes of cardiac arrhythmia or worsening of heart failure, based on the physiological signals. Example arrhythmia types include asystole, bradycardia, ventricular tachycardia, supraventricular tachycardia, wide complex tachycardia, atrial fibrillation, atrial flutter, ventricular fibrillation, atrioventricular block, premature ventricular contractions, and premature atrial contractions. The devices may store ECG and other physiological signal data collected during a time period including an episode as episode data. The devices may also store episode data quantifying the episodes, e.g., number and/or duration of episodes. The medical device may also store ECG and other physiological data for a time period as episode data in response to user input, e.g., from the patient or a caregiver.

### SUMMARY

In general, the disclosure describes techniques for triggering and/or prompting a patient to complete a survey for a clinical study related to an implantable medical device, such as an implantable cardiac monitor. In particular, this disclosure describes application-based approaches for collecting data related to a clinical study. An application-based clinical study may a cost-effective solution for gathering and managing data produced from the study. The application-based study may allow for a large number of patients to participate remotely. As such, the amount and quality of survey data collected via the techniques of this disclosure may provide real-time insights into study objectives.

Regularly scheduled patient surveys are designed to help clinical investigators understand the impact of clinical events in patients. However, clinical events of significant clinical interest may occur days or weeks before or after a regularly scheduled patient survey, which may allow for patients to forget important details or clinical events altogether. Lack of clarity and accuracy in patient surveys may impact the quality of analysis when part of a larger clinical study.

One purpose of a clinical study related to an implantable medical device is to leverage machine learning to evaluate the association between complex patterns of device-detected atrial fibrillation (AF) and other parameters and AF-related healthcare utilization, quality of life, AF-related symptoms, and adverse clinical outcomes in patients. In order to build a machine learning algorithm from clinical data sets, it is beneficial to recruit large cohorts of study participants, and may be additionally beneficial to obtain as close to real-time data from these participants as possible so that the data collected are accurate and timely. The problem then becomes how to obtain real-time data from participants in an app-based clinical study that will serve to inform the goals of the study.

Survey data collected via an application may provide accurate, real-time insights into study objectives. In accordance with the techniques of this disclosure, a device, such as a mobile phone, may be configured to trigger and prompt a patient to complete an in-application survey that may address one or more of medical and medication history, health-care utilization, and implantable medical device data experience impact. Moreover, the application may be configured to generate trigger-based reminders for medication updates. In addition, the application may be configured to utilize the location of patient in relation to a predefined geo-fenced area (e.g., an area near a study-related clinic, healthcare provider, and/or hospital) for triggering one or more surveys (e.g., health-care utilization surveys), which may for allow for surveys to be distributed and completed in a timely manner.

In one example of the disclosure, a method includes prompting a patient to complete a survey based on one or more of data received from an implantable medical device, a first time from an enrollment in a study related to the implantable medical device, a second time since a last survey, a medical event, or a detection of the patient in a geofenced area, receiving input from the patient in response to the survey, and sending the input from the patient to a database.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example medical device system configured to predict health events, and to respond to such predictions, in accordance with one or more techniques of this disclosure.

FIG. 2 is a block diagram illustrating an example configuration of the IMD of FIG. 1.

FIG. 3 is a conceptual side-view diagram illustrating an example configuration of the IMD of FIGS. 1 and 2.

FIG. 4 is a block diagram illustrating an example configuration of an external device that operates in accordance with one or more techniques of the present disclosure.

FIG. 5 is a block diagram illustrating an example computing system that operates in accordance with one or more techniques of the present disclosure.

FIG. 6 shows example data collected from an implantable medical device.

FIG. 7 shows example atrial fibrillation data plots.

FIG. 8 is a flowchart showing an example technique for prompting a survey.

FIG. 9 is a conceptual diagram showing an example patient medication survey user interface.

FIG. 10 is a conceptual diagram showing an example healthcare utilization survey user interface.

FIG. 11 is a conceptual diagram showing another example healthcare utilization survey user interface.

FIG. 12 is a conceptual diagram showing another example healthcare utilization survey user interface.

FIG. 13 is a conceptual diagram showing an example data impact and satisfaction survey user interface.

FIG. 14 illustrates example survey triggers from an implantable medical device.

FIG. 15 is a flowchart illustrating an example technique for prompting surveys.

FIG. 16 is a flowchart illustrating another example technique for prompting a survey.

Like reference characters refer to like elements throughout the figures and description.

### DETAILED DESCRIPTION

A variety of types of implantable and external medical devices detect arrhythmia episodes (e.g., atrial fibrillation) and other health events based on sensed ECGs and, in some cases, other physiological signals. External devices that may be used to non-invasively sense and monitor ECGs and other physiological signals include wearable devices with electrodes configured to contact the skin of the patient, such as patches, watches, or necklaces. Such external devices may facilitate relatively longer-term monitoring of patient health during normal daily activities.

Implantable medical devices (IMDs) also sense and monitor ECGs and other physiological signals, and detect health events such as arrhythmia episodes and worsening heart failure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

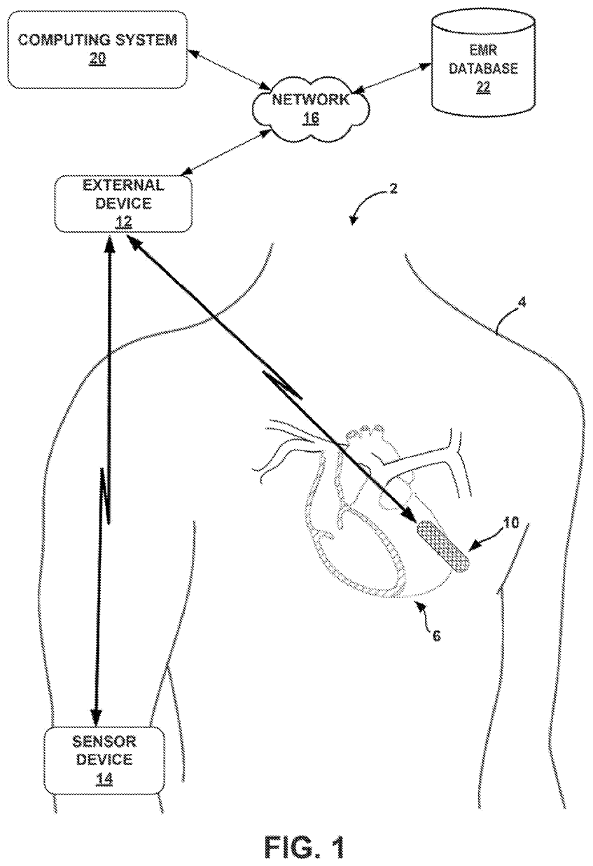

FIG. 1 is a block diagram illustrating an example medical device system 2 configured to predict health events of a patient 4, and to respond to such predictions, in accordance with the techniques of the disclosure. The example techniques may be used with an IMD 10, which may be in wireless communication with an external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense an ECG via the plurality of electrodes. In some examples, IMD 10 takes the form of the LINQ™ ICM. Although described primarily in the context of examples in which the IMD takes the form of an ICM, the techniques of this disclosure may be implemented in systems including any one or more implantable or external medical devices, including monitors, pacemakers, or defibrillators.

External device 12 is a computing device configured for wireless communication with IMD 10. External device 12 retrieves episode and other physiological data from IMD 10 that was collected and stored by IMD 10. In some examples, external device takes the form of a personal computing device of the patient or caregiver, such as a smart phone.

In the example illustrated by FIG. 1, system 2 also includes a sensor device 14 in wireless communication with external device 12. Sensor device 14 may include electrodes and other sensors to sense physiological signals of patient 4, and may collect and store physiological data and detect episodes based on such signals. In some examples, sensor device 14 is an external device wearable by patient 4. Sensor device 14 may be incorporated into the apparel of patient 14, such as within clothing, shoes, eyeglasses, a watch or wristband, a hat, etc. In some examples, sensor device 14 is a smartwatch or other accessory for a smartphone external device 12.

External device 12 retrieves episode and other physiological data from sensor device 14 that was collected and stored by sensor device 14. External device 12 may include a display and other user interface elements. In some examples, external device 12 presents physiological data retrieved from IMD 10 and/or sensor device 14, and/or statistical representations thereof, to patient 4 or another user. External device 12 may communicate with IMD 10 and/or sensor device 14 according to the Bluetooth® or Bluetooth® Low Energy (BLE) protocols, as examples.

External device 12 may be configured to communicate with a computing system 20 via a network 16. External device 12 may be used to retrieve data from IMD 10 and sensor device 14, and may transmit the data to computing system 20 via network 16. The retrieved data may include values of physiological parameters measured by IMD 10 and sensor device 14, data regarding episodes of arrhythmia or other health events detected by IMD 10 and sensor device 14, and other physiological signals or data recorded by IMD 10 sensor device 14. The data retrieved from IMD 10 and sensor device 14 may include values of various patient parameters, and/or may be used by computing system 20 to determine values of patient parameters. The values of patient parameters may be referred to as patient parametric data. Patient parametric data may be retrieved and or determined on a periodic basis to produce periodic values, e.g., on a daily basis to produce daily values.

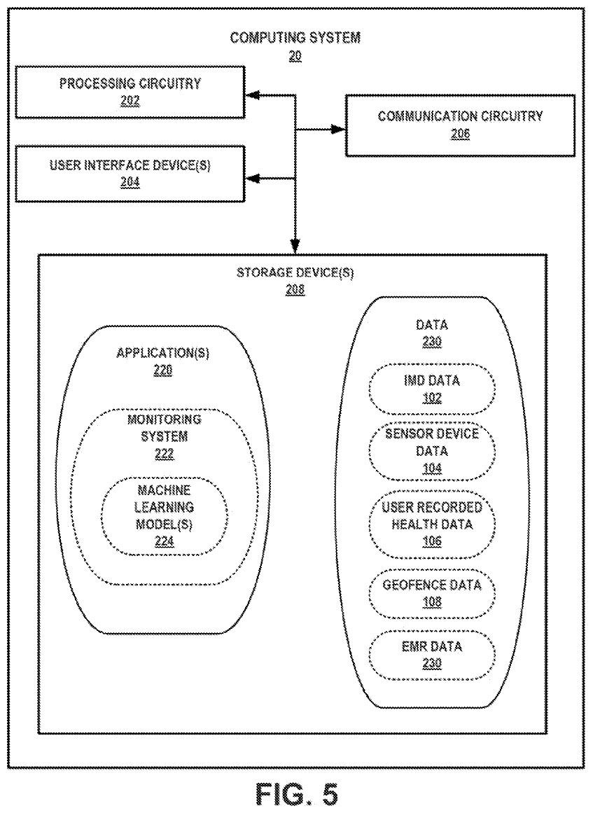

Computing system 20 may comprise computing devices configured to allow users, e.g., clinicians treating patient 4 and other patients, to interact with data collected from IMDs 10 and sensor devices 14 of their patients. In some examples, computing system 20 includes one or more handheld computing devices, computer workstations, servers or other networked computing devices. In some examples, computing system 20 may include one or more devices, including processing circuitry and storage devices, that implement a monitoring system 222 (FIG. 5). Monitoring system 222 may present parametric data of patients to clinicians to allow clinicians to remotely track and evaluate their patients. In some examples, monitoring system 222 may analyze the data and prioritize presentation of data or alerts for certain patients based on the analysis. Computing system 20, network 16, and monitoring system 222 may be implemented by the Medtronic Carelink™ Network, in some examples.

Network 16 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 16 may include one or more networks administered by service providers, and may thus form part of a large-scale public network infrastructure, e.g., the Internet. Network 16 may provide computing devices, such as computing system 20 and external device 12, access to the Internet, and may provide a communication framework that allows the computing devices to communicate with one another. In some examples, network 16 may be a private network that provides a communication framework that allows computing system 20 and external device 12 to communicate with one another but isolates one or more of these devices or data flows between these device from devices external to network 16 for security purposes. In some examples, the communications between computing system 20 and external device 12 are encrypted.

Computing system 20 may also retrieve data for patient 4 from electronic medical records (EMR) database 22. EMR database 22 may store electronic medical records, also referred to as electronic health records, for patient 4, which may be generated by various health care providers, laboratories, clinicians, insurance companies, etc. Although illustrated as a single database in FIG. 1, EMR database 22 may include various databases managed by various entities.

As examples, EMR database 22 may store a medication history of the patient, a surgical procedure history of the patient, a hospitalization history of the patient, emergency or urgent care visit history of the patient, scheduled clinic visit history of the patient, one or more lab or other clinical test results for patient 14, a cardiovascular history of patient 14, or co-morbidities of patient 14 such as atrial fibrillation, heart failure, or diabetes, as examples. As further examples, EMR database 22 may store medical images for patient 4, such as x-ray images, ultrasound images, echocardiograms, anatomical imagery, medical photographs, radiographic images, etc. The data stored in EMR database 22 may include the patient specific records for patient 4 and numerous other patients. In some examples, the data stored by EMR database 22 may include broader demographic information or population-type information for a plurality of patients.

Monitoring system 222, e.g., implemented by processing circuitry of computing system 20, may implement the techniques of this disclosure including developing an algorithm based on training sets of parametric data of a population of patients or subjects retrieved from IMDs 10 and external devices 14 of the population, and applying the algorithm to parametric data of an individual patient 4 to predict the occurrence of a clinically significant health event. In some examples, monitoring system trains one or more machine learning (ML) models for prediction of the health event. The output of the ML models for a particular patient may be a level of risk of the health event, a probability of the health event occurring within a certain time, and/or whether the risk or probability satisfies a threshold.

Example health events that may be predicted using the techniques of this disclosure include stroke, clinically significant AF requiring hospitalization or urgent care, and clinically significant episodes of syncope or dizziness. Parametric data that may be useful for predicting such health events may include cardiac rhythm data, such as heart rate data and data related to atrial fibrillation (AF) or other arrhythmia episodes. AF data may include quantifications of AF, referred to as AF burden, as well as patterns of AF burden over a plurality of periods of time. Parametric data that may be useful for predicting such clinically significant health events may additionally or alternatively include patient activity data or any other patient data or signals described herein.

Monitoring system 222 may also utilize data from EMR database 22 and/or data entered by the patient or a caregiver via external device 12 in conjunction with the parametric data from IMD 10 or sensor device 14. In some examples, data from EMR database 22 and/or data entered by the patient or caregiver may be used as inputs to the ML model(s) or other health event prediction algorithms implemented by monitoring system 222. In some examples, data from EMR database 22 and/or data entered by the patient or caregiver via external device 12 may provide classifications for training sets of parametric data from IMD 10 and sensor device 14 used to train one or more ML models to predict a health event. For example, data from EMR database 22 and/or data entered by the patient or caregiver via external device 12 may indicate whether, when, and to what degree of severity patient 4 experienced the clinically significant health event. Such data may be correlated with the parametric data to create a training set of parametric data. After an initial training phase, such training sets may be used for reinforcement learning and, in some cases, personalization of the one or more ML models.

Although the techniques are described herein as being performed by monitoring system 222, and thus by processing circuitry of computing system 20, the techniques may be performed by processing circuitry of any one or more devices or systems of a medical device system, such as computing system 20, external device 12, or IMD 10. The ML models may include, as examples, neural networks, deep learning models, convolutional neural networks, or other types of predictive analytics systems.

In one example of the disclosure, external device 12 may be configured to prompt patient 4 to complete one or more surveys based on data received from IMD 10 or other data related to patient 4. The surveys may be stored on external device 12 or may be accessed by external device 12 from computing system 20. As will be explained in more detail below, in a general example, external device 10 may be configured to prompt patient 4 to complete a survey based on one or more of data received from IMD 10, a first time from an enrollment in a study related to IMD 10, a second time since a last survey, a medical event, or a detection of patient 4 in a geofenced area. External device 12 may be further configured to receive input from patient 4 in response to the survey, and send the input from the patient to a database (e.g., EMR database 22).

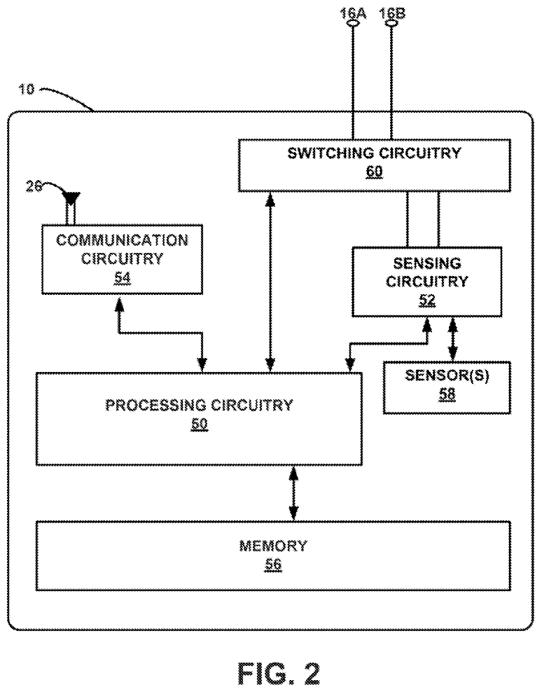

FIG. 2 is a block diagram illustrating an example configuration of IMD 10 of FIG. 1. As shown in FIG. 2, IMD 10 includes processing circuitry 50, sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor electrical activity of a heart of patient 4 of FIG. 1 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 56 as episode data for the detected arrhythmia episode. Processing circuitry 50 may also store parametric data in memory 56 including features of the ECG and data quantifying arrhythmia episodes, such as AF burden data.

Sensing circuitry 52 and/or processing circuitry 50 may be configured to detect cardiac depolarizations (e.g., P-waves of atrial depolarizations or R-waves of ventricular depolarizations) when the ECG amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and/or P-waves. Processing circuitry 50 may use the indications for determining features of the ECG including inter-depolarization intervals, heart rate, and heart rate variability. Sensing circuitry 52 may also provide one or more digitized ECG signals to processing circuitry 50 for analysis, e.g., for use in cardiac rhythm discrimination and/or to identify and delineate features of the ECG, such as QRS amplitudes and/or width, or other morphological features.

In some examples, sensing circuitry 52 measures impedance, e.g., of tissue proximate to IMD 10, via electrodes 16. The measured impedance may vary based on respiration and a degree of perfusion or edema. Processing circuitry 50 may determine parametric data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine parametric data, e.g., values of physiological parameters of patient 4, based on signals from sensors 58, which may be stored in memory 56.

In some examples, processing circuitry 50 transmits, via communication circuitry 54, the parametric and episode data for patient 4 to external device 12 of FIG. 1, which may transmit the data to network 16 for processing by monitoring system 222 of computing system 20. Communication circuitry 54 may include any suitable hardware, firmware, software or any combination thereof for communicating with another device, such as external device 12. Under the control of processing circuitry 50, communication circuitry 54 may receive downlink telemetry from, as well as send uplink telemetry to, external device 12 or another device with the aid of an internal or external antenna, e.g., antenna 26.

Although described herein in the context of example IMD 10, the techniques for cardiac arrhythmia detection disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, or a drug delivery device.

As discussed with respect to FIG. 1, sensor device 14 may be an external device such as a smartwatch, a fitness tracker, patch, or other wearable device. Sensor device 14 may be configured similarly to IMD 10 in the sense that it may include electrodes, sensors, sensing circuitry, processing circuitry, memory, and communication circuitry, and may function similarly to collect parametric data and communicate with external device 12. The sensors of and parametric data collected by IMD 10 and sensor device 14 may differ as described herein.

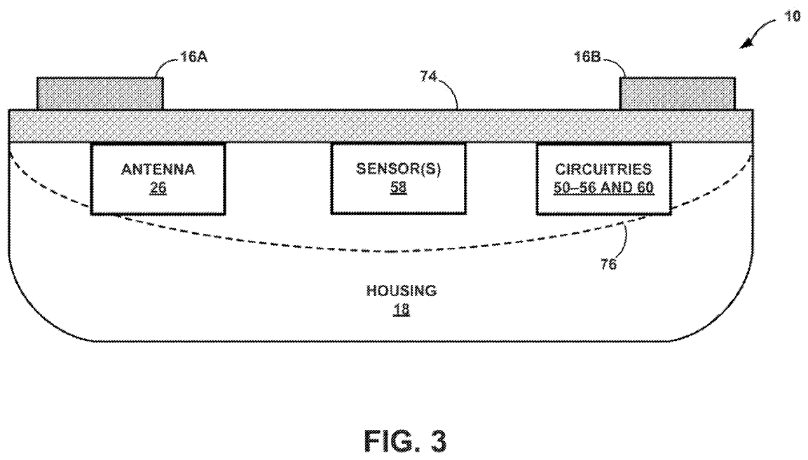

FIG. 3 is a conceptual side-view diagram illustrating an example configuration of IMD 10. In the example shown in FIG. 3, IMD 10 may include a leadless, subcutaneously-implantable monitoring device having a housing 18 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 18. In the illustrated example, antenna 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 18 such that housing 18 and cover 74 enclose antenna 26, sensors 58, and circuitries 50-56 and 60, and protect the antenna and circuitries from fluids such as body fluids.

One or more of antenna 26, sensors 58, or circuitries 50-56 may be formed on insulative cover 74, such as by using flip-chip technology. Insulative cover 74 may be flipped onto a housing 18. When flipped and placed onto housing 18, the components of IMD 10 formed on the inner side of insulative cover 74 may be positioned in a gap 76 defined by housing 18. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

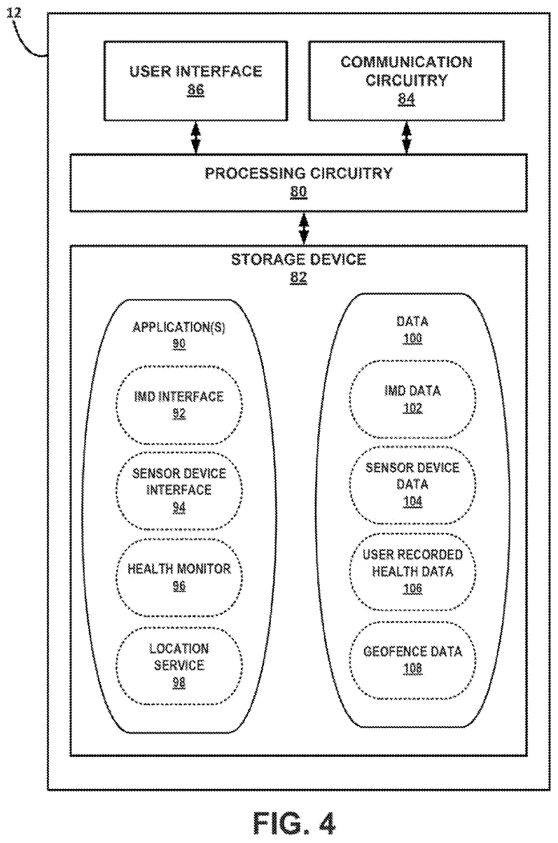

FIG. 4 is a block diagram illustrating an example configuration of external device 12. In some examples, external device 12 takes the form of a mobile device, such as a mobile phone, a “smart” phone, a laptop, a tablet computer, or a personal digital assistant (PDA). As shown in the example of FIG. 4, external device 12 includes processing circuitry 80, storage device 82, communication circuitry 84, and a user interface 86. Although shown in FIG. 4 as a stand-alone device for purposes of example, external device 12 may be any component or system that includes processing circuitry or other suitable computing environment for executing software instructions and, for example, need not necessarily include one or more elements shown in FIG. 4 (e.g., in some examples components such as storage device 82 may not be co-located or in the same chassis as other components).

Processing circuitry 80, in one example, is configured to implement functionality and/or process instructions for execution within external device 12. For example, processing circuitry 80 may be capable of processing instructions, including applications 90, stored in storage device 82. Examples of processing circuitry 80 may include, any one or more of a microprocessor, a controller, a digital signal processor (DSP), an application specific integrated circuit (ASIC), a field-programmable gate array (FPGA), or equivalent discrete or integrated logic circuitry.

Storage device 82 may be configured to store information within external device 12, including applications 90 and data 100. Storage device 82, in some examples, is described as a computer-readable storage medium. In some examples, storage device 82 includes a temporary memory or a volatile memory. Examples of volatile memories include random access memories (RAM), dynamic random access memories (DRAM), static random access memories (SRAM), and other forms of volatile memories known in the art. Storage device 82, in one example, is used by applications 90 running on external device 12 to temporarily store information during program execution. Storage device 82, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

External device 12 utilizes communication circuitry 84 to communicate with other devices, such as IMD 10, sensor device 14, and computing system 20 of FIG. 1. Communication circuitry 84 may include a network interface card, such as an Ethernet card, an optical transceiver, a radio frequency transceiver, or any other type of device that can send and receive information. Other examples of such network interfaces may include 3G, 4G, 5G, and WiFi radios.

External device 12 also includes a user interface 86. User interface 86 may be configured to provide output to a user using tactile, audio, or video stimuli and receive input from a user through tactile, audio, or video feedback. User interface 86 may include, as examples, a presence-sensitive display, a mouse, a keyboard, a voice responsive system, video camera, microphone, or any other type of device for detecting a command from a user, a sound card, a video graphics adapter card, or any other type of device for converting a signal into an appropriate form understandable to humans or machines, a speaker, a cathode ray tube (CRT) monitor, a liquid crystal display (LCD), or any other type of device that can generate intelligible output to a user. In some examples, a presence-sensitive display includes a touch-sensitive screen.

Example applications 90 executable by processing circuitry 80 of external device 12 include an IMD interface application 92, a sensor device interface application 94, a health monitor application 96, and a location service 98. Execution of IMD interface 92 by processing circuitry 80 configures external device 12 to interface with IMD 10. For example, IMD interface 92 configures external device 12 to communicate with IMD 10 via communication circuitry 84. Processing circuitry 80 may retrieve IMD data 102 from IMD 10, and store IMD data 102 in memory 82. IMD interface 92 also configures user interface 86 for a user to interact with IMD 10 and/or IMD data 102. For example, IMD interface 92 configures external device 12 to communicate with IMD 10 via communication circuitry 84. Processing circuitry 80 may retrieve IMD data 102 from IMD 10, and store IMD data 102 in memory 82. IMD interface 92 also configures user interface 86 for a user to interact with IMD 10 and/or IMD data 102. Similarly, sensor device interface 94 configures external device 12 to communicate with sensor device 14 via communication circuitry 84, retrieve sensor device data 104 from sensor device 14, and store sensor device data 104 in memory 82. Sensor device interface 42 also configures user interface 86 for a user to interact with sensor device 14 and/or sensor device data 104.

Health monitor 96 may be configured facilitate monitoring the health of patient 4 by a user, such as the patient or a caregiver. Health monitor 96 may present health information, such as at least portions of IMD data 102 and/or sensor device data 104, via user interface 86. Health monitor 96 may also collect information regarding the patient's health from the user via user interface 86, and store the information as user recorded health data 106. In some examples, health monitor 96 present the user with a questionnaire or survey seeking health data 106 from the user. Health monitor 96 may present the surveys according to a schedule, in response to IMD data 102 and/or sensor device data 104 indicating that patient 4 experienced a health event, and/or based on a location of patient 4, e.g., in response to location service 98 indicating that patient 4 entered a geofence area defined by geofence data 108. Presenting surveys in response to health events may facilitate timely capture of user recorded health data 106 regarding the health event. In some examples, geofence areas are defined around clinics, hospitals, or the like, and entry into a such geofence area may similarly indicate that patient 4 experienced a health event meriting timely collection of user recorded health data 106. Processing circuitry 80 may also store the times and durations of patient entering a geofence area as geofence data 108.

IMD data 102 and sensor device data 104 may include patient parametric data derived from sensed physiological signals as described herein. As examples, IMD data 102 may include periodic (e.g., daily) values of one or more of: heart rate, heart rate variability, one or more ECG morphological features or intrabeat intervals, AF and/or other arrhythmia burden (e.g., number, time, or percent time per period), respiratory rate, perfusion, and activity levels.

As examples, sensor device data 104 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

As examples, user recorded health data 106 may include one or more of: exercise and activity data, sleep data, symptom data, quality of life data, nutrition data, medication taking or compliance data, allergy data, weight, and height. Sensor device data 104 and/or user recorded health data 106 may include one or more of the types of data listed in Table 1 below.

TABLE 1

| Data Type | Relevance |
|---|---|
| Health Records (Allergies) | Could help explain why certain |
| | medications were not used |
| Health Records (Conditions) | Confirms patient medical conditions |
| Health Records (Lab results) | Confirm disease states |
| Health Records (Medications) | Confirm AF medication use |
| | (AAD, OAC, etc.) |
| Health Records (Procedures) | Tracks interventions that |
| | could impact AF |
| Health Records (Vital Signs) | Compare to IMD data |
| Activity | Compare to IMD data |
| Sex | |
| Date of Birth | |
| Walking + Running Distance | Compare to IMD data |
| Resting Energy | Compare to IMD data |
| Active Energy | Compare to IMD data |
| Exercise Minutes | Compare to IMD data |
| Stand Hour | Compare to IMD data |
| Stand Time | Compare to IMD data |
| Height | |
| Body Mass | |
| Body Mass Index | |
| Lean Body Mass | Greater understanding of body comp |
| | and fitness level compared to BMI |
| Body Fat Percentage | Greater understanding of body comp |
| | and fitness level compared to BMI |
| Waist Circumference | Greater understanding of body comp |
| | and fitness level compared to BMI |
| Heart Rate | Compare to IMD data |
| Low Heart Rate Notifications | Compare to IMD data |
| High Heart Rate Notifications | Compare to IMD data |
| Irregular Rhythm Notifications | Compare to IMD data |
| Resting Heart Rate | Compare to IMD data |
| Heart Rate Variability | Compare to IMD data |
| Walking Heart Rate Average | Compare to IMD data |
| Heart Beat Series | Compare to IMD data |
| ElectroCardiograms (ECG) | Compare to IMD data |
| Blood Oxygen | Can offer greater insight into |
| | COPD/HF/apnea influenced AF |
| Blood Pressure | Indicator of cardiovascular risk |
| Systolic Blood Pressure | Indicator of cardiovascular risk |
| Diastolic Blood Pressure | Indicator of cardiovascular risk |
| Respiratory Rate | Indicator of potential HF or COPD |
| | conditions that can impact AF |
| VO2 Max | Indicator of physical fitness, HIGHLY |
| | predictive of clinical outcomes |
| Blood Glucose | Indicates Diabetes management, |
| | driver of CHADS score and impacts |
| | clinical outcomes in AF patients |
| Insulin Delivery | Indicates Diabetes management, |
| | driver of CHADS score and impacts |
| | clinical outcomes in AF patients |
| Peripheral Perfusion Index | Indicator of HF or COPD |
| | that can influence AF |
| Sleep | Sleep patterns may influence AF |
| Dietary Energy | Can be associated with AF |
| Carbohydrates | Can be associated with AF |
| Fiber | Can be associated with AF |
| Dietary Sugar | Can be associated with AF |
| Total Fat | Can be associated with AF |
| Monounsaturation Fat | Can be associated with AF |
| Polyunsaturated Fat | Can be associated with AF |
| Saturated Fat | Can be associated with AF |
| Cholesterol | Can be associated with AF |
| Protein | Can be associated with AF |
| Calcium | Can be associated with AF |
| Potassium | Can be associated with AF |
| Sodium | Can be associated with AF |
| Caffeine | Can be associated with AF |
| Dizziness | Can be associated with AF |
| Fainting | Can be associated with AF |
| Fatigue | Can be associated with AF |
| Chest Tightness or Pain | Can be associated with AF |
| Raid, Pounding, or Fluttering | Can be associated with AF |
| Heartbeat | |
| Shortness of Breath | Can be associated with AF |
| Skipped Heartbeat | Can be associated with AF |
| Headache | Can be associated with AF |
| Night Sweats | Can be associated with AF |
| Sleep Changes | Can be associated with AF |

As will be described in more detail below, in one example of the disclosure, external device 12 may be configured to prompt patient 4 to complete one or more surveys based on data received from IMD 10 or other data related to patient 4. The surveys may be stored on external device 12 or may be accessed by external device 12 from computing system 20. As will be explained in more detail below, in a general example, external device 10 may be configured to prompt patient 4 to complete a survey based on one or more of data received from IMD 10, a first time from an enrollment in a study related to IMD 10, a second time since a last survey, a medical event, or a detection of patient 4 in a geofenced area. External device 12 may be further configured to receive input from patient 4 in response to the survey, and send the input from the patient to a database (e.g., EMR database 22).

FIG. 5 is a block diagram illustrating an example configuration of computing system 20. In the illustrated example, computing system 24 includes processing circuitry 202 for executing applications 220 that include monitoring system 222 or any other applications described herein. Computing system 20 may be any component or system that includes processing circuitry or other suitable computing environment for executing software instructions and, for example, need not necessarily include one or more elements shown in FIG. 5 (e.g., user interface devices 204, communication circuitry 206; and in some examples components such as storage device(s) 208 may not be co-located or in the same chassis as other components). In some examples, computing system 20 may be a cloud computing system distributed across a plurality of devices.

In the example of FIG. 5, computing system 24 includes processing circuitry 202, one or more user interface (UI) devices 204, communication circuitry 206, and one or more storage devices 208. Computing system 20, in some examples, further includes one or more application(s) 220 such as monitoring system 222, that are executable by computing system 20.

Processing circuitry 202, in one example, is configured to implement functionality and/or process instructions for execution within computing system 20. For example, processing circuitry 202 may be capable of processing instructions stored in storage device 208. Examples of processing circuitry 202 may include any one or more of a microprocessor, a controller, a digital signal processor (DSP), an application specific integrated circuit (ASIC), a field-programmable gate array (FPGA), or equivalent discrete or integrated logic circuitry.

One or more storage devices 208 may be configured to store information within computing device 20 during operation. Storage device 208, in some examples, is described as a computer-readable storage medium. In some examples, storage device 208 is a temporary memory, meaning that a primary purpose of storage device 208 is not long-term storage. Storage device 408, in some examples, is described as a volatile memory, meaning that storage device 408 does not maintain stored contents when the computer is turned off. Examples of volatile memories include random access memories (RAM), dynamic random access memories (DRAM), static random access memories (SRAM), and other forms of volatile memories known in the art. In some examples, storage device 208 is used by software or applications 220 running on computing system 20 to temporarily store information during program execution.

Storage devices 208 may further be configured for long-term storage of information, such as applications 220 and data 230. In some examples, storage devices 208 include non-volatile storage elements. Examples of such non-volatile storage elements include magnetic hard discs, optical discs, floppy discs, flash memories, or forms of electrically programmable memories (EPROM) or electrically erasable and programmable memories (EEPROM).

Computing system 20, in some examples, also includes communication circuitry 206 to communicate with other devices and systems, such as IMD 10 and external device 12 of FIG. 1. Communication circuitry 206 may include a network interface card, such as an Ethernet card, an optical transceiver, a radio frequency transceiver, or any other type of device that can send and receive information. Other examples of such network interfaces may include 3G, 4G, 5G, and WiFi radios.

Computing system 20, in one example, also includes one or more user interface devices 204. User interface devices 204, in some examples, may be configured to provide output to a user using tactile, audio, or video stimuli and receive input from a user through tactile, audio, or video feedback. User interface devices 204 may include, as examples, a presence-sensitive display, a mouse, a keyboard, a voice responsive system, video camera, microphone, or any other type of device for detecting a command from a user, a sound card, a video graphics adapter card, or any other type of device for converting a signal into an appropriate form understandable to humans or machines, a speaker, a cathode ray tube (CRT) monitor, a liquid crystal display (LCD), or any other type of device that can generate intelligible output to a user.

Applications 220 may also include program instructions and/or data that are executable by processing circuitry 202 of computing system 20 to cause computing system 20 to provide the functionality ascribed to it herein. Example application(s) 220 may include monitoring system 22. Other additional applications not shown may alternatively or additionally be included to provide other functionality described herein and are not depicted for the sake of simplicity.

In accordance with the techniques of the disclosure, computing system 20 receives IMD data 102, sensor device data 104, user recorded health data 106, and geofence data 108 from external device 12 via communication circuitry 206. Processing circuitry 202 stores these as data 230 in storage devices 208.

Computing system 20 may also receive EMR data 230 from EMR database 22 (FIG. 1) vis communication circuitry 206, and store EMR data 230 in storage device 208. EMR data 230 may include, for each of a plurality of patients or subjects a medication history, a surgical procedure history, a hospitalization history, emergency or urgent care visit history, scheduled clinic visit history, one or more lab or other clinical test results, a procedure history, a cardiovascular history, or co-morbidities such as atrial fibrillation, heart failure, syncope, or diabetes, as examples. As further examples, EMR data 230 may include medical images, such as x-ray images, ultrasound images, echocardiograms, anatomical imagery, medical photographs, radiographic images, etc.

Monitoring system 222, e.g., implemented by processing circuitry of computing system 20, may implement the techniques of this disclosure including developing an algorithm based on training sets of parametric data, e.g., from IMD data 102 and sensor device data 104, and in some cases user recorded health data 106 and EMR data 230, of a population of patients or subjects, and applying the algorithm to parametric data of an individual patient 4 to predict the occurrence of a clinically significant health event. In some examples, monitoring system 222 trains one or more machine learning (ML) models 224 for prediction of the health event. The output of the ML models for a particular patient may be a level of risk of the health event, a probability of the health event occurring within a certain time, and/or whether the risk or probability satisfies a threshold.

In some examples, data from EMR database 22 and/or data entered by the patient or caregiver via external device 12 may provide classifications for training sets of parametric data from IMD 10 and sensor device 14 used to train one or more ML models to predict a health event. For example, data from EMR database 22 and/or data entered by the patient or caregiver via external device 12 may indicate whether, when, and to what degree of severity patient 4 experienced the clinically significant health event. Such data may be correlated with the parametric data to create a training set of parametric data. After an initial training phase, such training sets may be used for reinforcement learning and, in some cases, personalization of the one or more ML models.

Although the techniques are described herein as being performed by monitoring system 222, and thus by processing circuitry of computing system 20, the techniques may be performed by processing circuitry of any one or more devices or systems of a medical device system, such as computing system 20, external device 12, or IMD 10. The ML models may include, as examples, neural networks, deep learning models, convolutional neural networks, or other types of predictive analytics systems.

Techniques for triggering and prompting patients to complete a medical survey will now be described. The techniques of this disclosure are described with reference to external device 12 prompting and delivering surveys, but it should be understood that any combination of computing system 20, external device 12, IMD 10, and/or sensor device 14 may be configured to perform one or more of the techniques of this disclosure.

Regularly scheduled patient surveys are designed to help clinical investigators understand the impact of clinical events in patients. However, clinical events of significant clinical interest may occur days or weeks before or after a regularly scheduled patient survey, which may allow for patients to forget important details or clinical events altogether. Lack of clarity and accuracy in patient surveys may impact the quality of analysis when part of a larger clinical study.

One purpose of a clinical study related to IMD 10 is to leverage machine learning to evaluate the association between complex patterns of device-detected atrial fibrillation (AF) (e.g., detected by IMD 10) and other parameters and AF-related healthcare utilization, quality of life, AF-related symptoms, and adverse clinical outcomes in patients. In order to build a machine learning algorithm from clinical data sets, it is beneficial to recruit large cohorts of study participants, and may be additionally beneficial to obtain as close to real-time data from these participants as possible so that the data collected are accurate and timely. The problem then becomes how to obtain real-time data from participants in an app-based clinical study that will serve to inform the goals of the study.

An application-based clinical study (e.g., health monitor 96 operating on external device 12 and/or computing system 20) may be a cost-effective solution that will allow for a large number of patients (e.g., patient 4 of FIG. 1) to participate remotely. Survey data collected via an application (e.g., health monitor 96) executed by external device 12 may provide accurate, real-time insights into study objectives. In accordance with the techniques of this disclosure, health monitor 96 may be configured to trigger and prompt patient 4 to complete an in-application survey that may address one or more of medical and medication history, health-care utilization, and IMD (e.g., IMD 10) data experience impact. Moreover, health monitor 96 may be configured to generate trigger-based reminders for medication updates. In addition, health monitor 96 may be configured to utilize the location of patient 4 in relation to a predefined geo-fenced area (e.g., an area near a study-related clinic, healthcare provider, and/or hospital) for triggering one or more surveys (e.g., health-care utilization surveys), which may for allow for surveys to be distributed and completed in a timely manner.

In one example of the disclosure, external device 12 may be configured to receive data from IMD 10. The external device 12 (e.g., executing health monitor 96) may be configured to prompt patient 4 to complete a survey based on data received from IMD 10. In general, external device 12 may be configured to trigger a survey for patient 4 to complete when the data received from IMD 10 indicates that a clinical event of interest has occurred. In one example, IMD 10 is an implantable cardiac monitor. In this example, a clinical event of interest may include an atrial fibrillation event and/or data indicative of atrial fibrillation burden. Of course, the techniques of this disclosure for triggering and prompting surveys may be used with other types of external or implantable medical devices and may be used with any type of event of clinical interest.

In one example of the disclosure, health monitor 96 may be configured to prompt patient 4 to complete a survey based on data received from IMD 10. External device 12 may prompt patient 4 using one or more different techniques. In one example, external device 12 may send patient 4 an e-mail and/or text message. The e-mail, text, or other notification may include a link to a website or application (e.g., health monitor 96) that will display the survey and collect the patient feedback. In other examples, external device 12 may cause one or more of a banner notification, application notification, audio notification, and/or haptic notification to be initiated on external device 12, wherein the notifications indicate that there is a survey waiting to be completed in an application (e.g., health monitor 96) related to IMD 10. In other examples, external device 12 may cause the survey to be automatically displayed on external device 12 or automatically displayed when the application (e.g., health monitor 96) related to IMD 10 is executed (e.g., opened by patient 4) on external device 12.

External device 12 may be configured to receive input from patient 4 in response to the survey. For example, external device 12 may display a user interface that allows patient 4 to input answers related to the survey questions. The survey may be in the form of a text box, selectable buttons, dropdown menus, or other forms of data input. After receiving the input from the patient, external device 12 may send the input from the patient to a database (e.g., computing system 20 and/or EMR database 22 of FIG. 1). By using the medical device data collected from the patient (e.g., data from IMD 10) to dictate when a clinical event of interest may have occurred and to trigger the patient survey to be sent to the patient via a mobile app, the techniques of this disclosure may reduce the latency between an event and the subsequent survey intended to capture information about that clinical event.

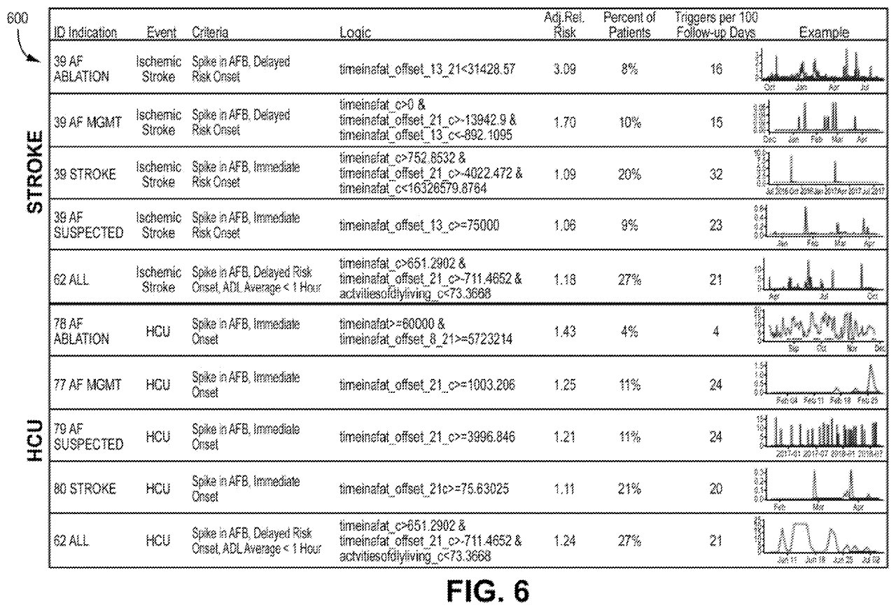

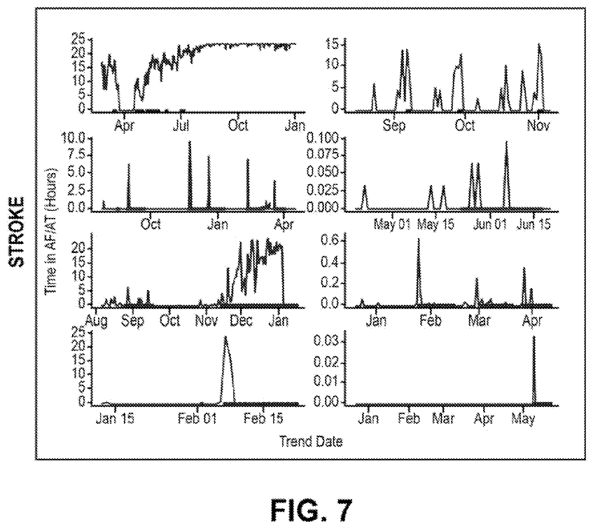

FIG. 6 shows example data 600 collected by IMD 10 that may be indicative of a clinical event of interest related to atrial fibrillation (AF) and/or other conditions monitored by IMD 10. Data 600 includes data from IMD 10 that may be indicative of one or more of ischemic stroke and/or a higher incidence of health care utilization (HCU). FIG. 7 shows example AF data plots 700 specific to stroke risks of patients indicated for stroke, suspected AF, AF ablation, and AF management.

External device 12, computing system 20, and/or another device may be configured to continuously monitor IMD 10 of patient 4, either locally or through routine data transmission, for significant clinical events (e.g., data trends, specific clinical moments, data associations). Once the event of interest occurs, external device 12 may be configured to prompt patient 4 to complete a specified patient survey or questionnaire through an application (e.g., health monitor 96) to collect relevant information from the patient via their mobile device (e.g., external device 12). Patient 4 answers to survey questions may be collected and recorded for statistical analysis. This approach would result in more accurate information from patients and could potentially have large impacts on budgets, timelines, and clinical insights for clinical trials.

In the above example, surveys may be administered through an application (e.g., health monitor 96) executed by external device 12, where the application is related to a clinical study related to health conditions monitored by IMD 10. Health monitor 96 may be built on an operating system for a mobile device. In addition to triggering surveys based on data from IMD 10, a research platform built within this application may be configured to control the frequency of surveys to ensure real-time data are obtained. In this context, real-time data may be considered data that is contemporaneous with clinical events of interest, device triggers, adverse health episodes, clinical visits, etc. Having contemporaneous, real-time data better ensures that accurate and/or useful data is captured to be used in the clinical study and/or patient treatment decisions.

In some examples, the application (e.g., health monitor 96) executed by external device 12 may be configured to provide for remote enrollment in a study and follow-up with patients by administering an electronic consent. In some examples, IMD 10 may be configured to collect and report measured parameters (e.g., cardiac parameters) through another network (e.g., the Carelink network), regardless of whether or not the patient is enrolled in the study.

External device 12 may be configured to administer various types of patient surveys to assess quality of life, healthcare utilization, clinical events, changes in medical management, symptoms related to IMD 10 (e.g., AF and/or AFB), and medication use and compliance. External device 12 may also be configured to administer surveys to evaluate patient preferences for viewing and interacting with data from IMD 10. In addition, external device 12 may also collect data related to the use of the application (e.g., time in app, in-app feature use, number of clicks, etc.). In some examples, the exact content of the surveys may change throughout the study.

As described above, external device 12 may be configured to prompt patients to complete surveys through an application (e.g., health monitor 96) executed by external device 12. In some example, external device 12 may be configured to prompt surveys at variable times based on one or more of the following factors: time from enrollment in a study, time since last survey was completed, time since last survey was prompted, device data from IMD 10, clinical events, a single AF episode lasting longer than a predetermined threshold (e.g., 1-hour), cumulative daily AF burden greater than a predetermined threshold (e.g., 5% or greater), location of a patient with a predetermined geofence area, location of a in a geofence longer than a threshold time (e.g., ≥45 minutes), and other factors.

In some examples, using two or more factors to trigger and prompt a survey may allow for more useful information to be gathered from a patient at times that are either contemporaneous with a clinical event of interest and/or contemporaneous with a healthcare utilization event (e.g., a planned or unplanned visit to a clinic, hospital, or healthcare provider). For example, health monitor 96 may use position/location capabilities of the device to determine a location of patient 4. If patient 4 goes within an area near clinic, hospital, or healthcare provider (e.g., a geofenced area), a survey may be prompted, as location within the geofenced area may be indicative of a healthcare utilization event. The geofenced area may be any predefined boundaries around clinic, hospital, or healthcare provider. In some example, health monitor 96 may only trigger a survey in situations where the patient is within a geofenced area longer than a predetermined time. This may avoid situations where a patient merely passes by a geofenced area, which may not be indicative of a healthcare utilization event.

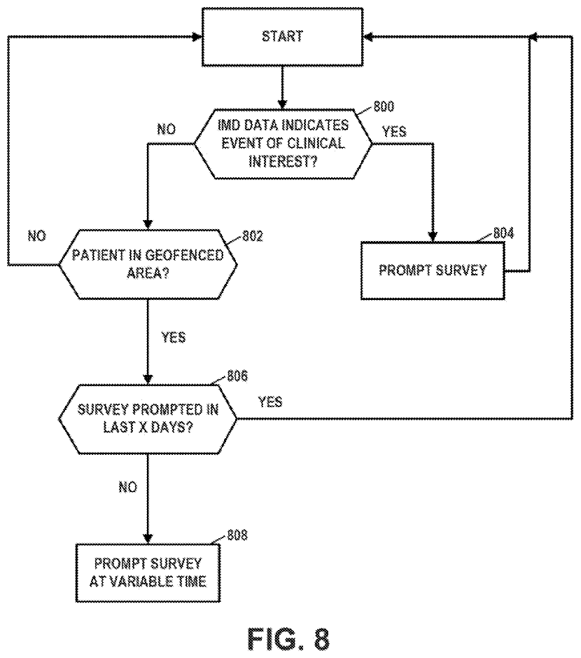

FIG. 8 is a flowchart showing an example technique for prompting a survey. In the example of FIG. 8, health monitor 96 analyses IMD data 102 received from IMD 10 and determines if the IMD data indicates an event of clinical interest (800). If yes, health monitor 96 prompts patient 4 to complete a survey (804). In this example, health monitor 96 prompts a survey immediately for any event of clinical interest. In other examples, as will be discussed below, health monitor 96 may prompt the survey based both on clinical events of interest and a time since a last survey was completed in order to avoid overburdening the patient with too many surveys. Furthermore, each type of clinical event of interest may trigger different surveys at different frequencies.

If health monitor 96 determines that the IMD data does not indicate an event of clinical interest (NO branch of 800), health monitor 96 may then determine if the patient is in a geofenced area (802). If no, health monitor 96 may return to the start and scan for additional IMD data. If yes, health monitor 96 may then determine if a survey has been prompted in the last X (e.g., 7) number of days (806). If yes, health monitor 96 may return to the start and scan for additional IMD data. If no, health monitor 96 may prompt a survey at a variable time (e.g., a random time in the next X number of days) (808). The techniques of FIG. 8 are just one example. Any number or combination of triggers may be used to prompt surveys in accordance with the techniques of this disclosure.

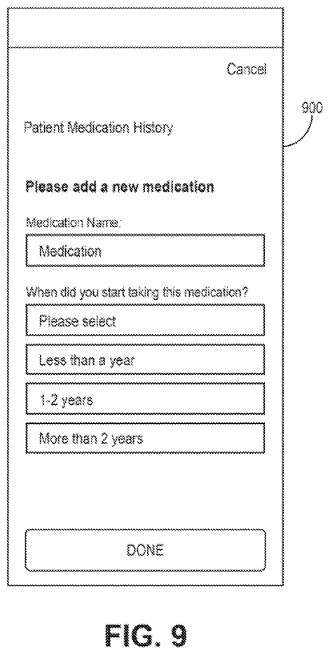

FIG. 9 is a conceptual diagram showing an example patient medication survey user interface (UI). UI 900 that collects survey data from patient 4 concerning medications being taken. UI 900 collects the survey data using selectable buttons as well as text entry boxes.

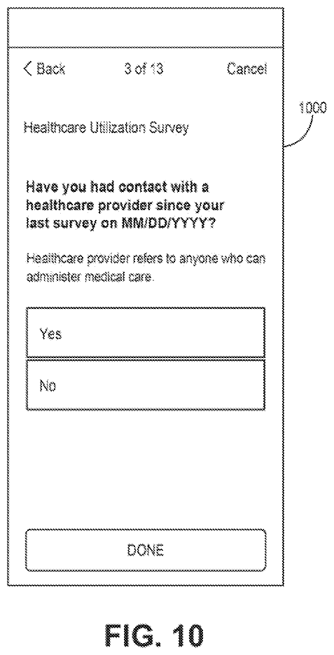

FIG. 10 is a conceptual diagram showing an example healthcare utilization survey user interface. UI 1000 that collects survey data from patient 4 concerning contact with a healthcare provider since the last survey. UI 1000 collects survey data using selectable buttons.

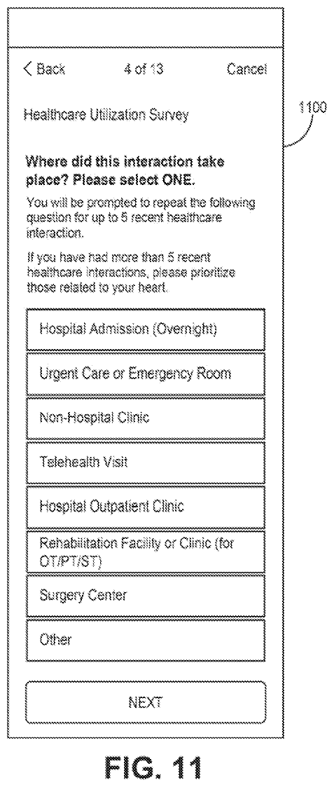

FIG. 11 is a conceptual diagram showing another example healthcare utilization survey user interface. UI 1100 that collects survey data from patient 4 concerning more specific information regarding an interaction with a healthcare provider. UI 1100 collects survey data using selectable buttons.

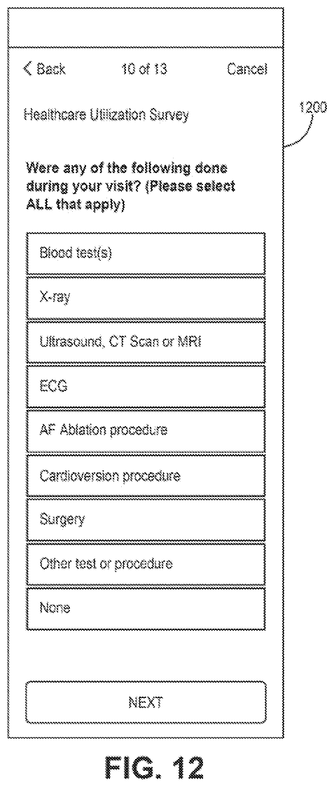

FIG. 12 is a conceptual diagram showing another example healthcare utilization survey user interface. UI 1200 that collects survey data from patient 4 concerning procedures performed during the last interaction with a healthcare provider. UI 1200 collects survey data using selectable buttons.

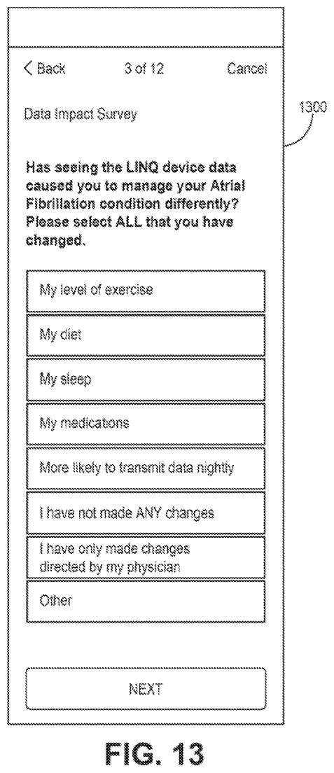

FIG. 13 is a conceptual diagram showing an example data impact and satisfaction survey user interface. UI 1300 that collects survey data from patient 4 concerning patient behavior based on viewing data from IMD 10. UI 1300 collects survey data using selectable buttons.

Survey responses are captured by health monitor 96 by pushing notification to patient 4 and having tasks presented on the tasks main view screen of the UI. Once patient 4 has completed a survey, health monitor 96 may store the response in memory. Computing system 20 and/or EMR database 22 may pull the data for analysis (e.g., by the clinical study administrator).

In some examples, health monitor 96 is configured to deliver a set of surveys (e.g., more than one survey) to patient 4. Health monitor 96 may deliver a portion of these surveys at regular intervals (e.g., monthly, every 3 months, etc.). Health monitor 96 may use more complex triggers and prompting logic for other surveys in a study (e.g., IMD data/symptoms surveys and healthcare utilization surveys). For these surveys, health monitor 96 may identify if patient 4 has experienced a qualifying trigger event (e.g., a clinical event of interest) to prompt a survey. In another example, health monitor 96 may identify if patient 4 has entered a geofence boundary of a Hospital, Urgent Care Clinic, or other healthcare provider (e.g., via location sharing and connection with a Geofence service) to prompt a survey. Health monitor 96 may be configured to schedule surveys in a manner such that patient 4 is not overburdened with excess surveys, and that the surveys are spaced and delivered with a degree of randomness that keeps the surveys novel from the viewpoint of patient 4. In this way, patient 4 is less likely to skip the surveys as they will appear to be novel and not routine.

In general, health monitor 96 may operate an algorithm that determines when to appropriately deliver surveys at a regular interval, and prevent excessive deployment of surveys in response to a triggering event. For example, a Healthcare Utilization Survey may be deployed when the health monitor 96 identifies that a patient has been experiencing significant atrial fibrillation consistently over several days. In this situation, health monitor 96 will not prompt patient 4 to complete a survey every day, but rather, the first day the AF event qualifies as a trigger, and at a regular schedule thereafter.

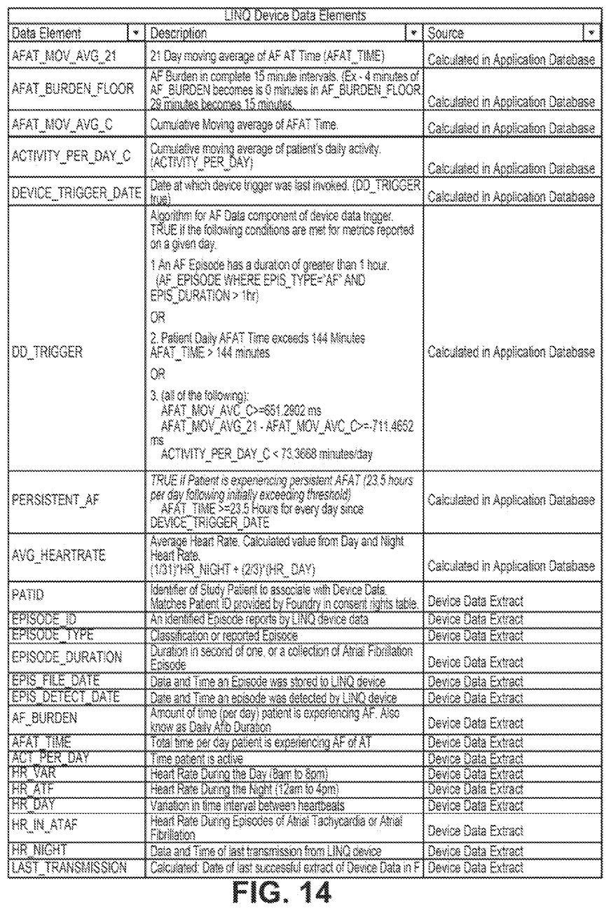

Health monitor 96 may collect patient data metrics from internal sources and external sources (e.g., IMD 10, EMR database 22, computing system 20, and/or sensor device 14), may perform a calculation to determine if patient 4 had a qualifying clinical event of interest (e.g., a qualifying AF Event). FIG. 14 illustrates example survey triggers received from IMD 10. For example, the equation defined in column DD TRIGGER shown in FIG. 14 uses data about the record Atrial fibrillation of patient 4 to determine if patient 4 is experiencing a qualifying event. Health monitor 96 may also integrate with a geofence location service, which may parse location information of external device 12 into geofence events, to determine if patient 4 has come within a defined range of a healthcare entity (e.g., Hospital, Urgent Care clinic, or other healthcare provider).

Health monitor 96 may evaluate both the device data and geofence data, along with other information available to health monitor 96 that is associated with a patient's participation in the study (e.g., last survey completion date, survey send date, etc.) to evaluate whether to deploy a survey in response to a qualifying event.

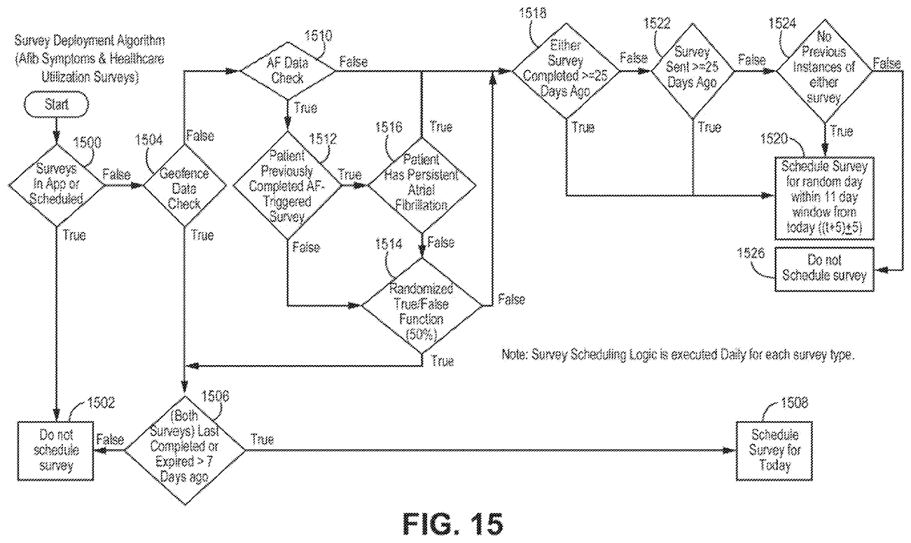

FIG. 15 is a flowchart illustrating an example technique for prompting surveys. Health monitor 96 may use the techniques of FIG. 15 for determining whether or not to prompt a survey for both an AF symptoms survey and a healthcare utilization survey. In some examples, health monitor 96 may perform the techniques of FIG. 15 daily for each type of survey. Of course, the techniques of FIG. 15 may be used with other frequencies, other IMDs, other health conditions, and with other types of surveys.

Health monitor 96 may first determine if there are surveys already scheduled or available to the patient (1500). If true, health monitor 96 will not prompt further surveys (1502). If health monitor determines that there are not surveys already scheduled or available to the patient, health monitor 96 will then determine if the patient is within a geofenced area (1504). If true, health monitor 96 will determine if both the AF symptoms and healthcare utilization surveys have been completed or expired in the last 7 days (1506). If false, health monitor 96 will not prompt further surveys (1502). If true, health monitor 96 will prompt a survey for today (1508).

If health monitor 96 determines that the patient is not in the geofenced area, health monitor 96 will then perform an AF data check (1510). The AF data check may include analyzing IMD data received from IMD 10. If the AF data check indicates an event of clinical interest (e.g., an AF event) has occurred, health monitor 96 may then determine if the patient has previously completed an AF-triggered survey (1512). If false, health monitor 96 will randomly (1514) proceed to either branch 1506 or 1518 to determine the frequency and timing of the AF-triggered survey.

If health monitor 96 determines that the patient has previously completed an AF-triggered survey, health monitor 96 will then determine if the patient has persistent AF (1516). If false, health monitor 96 will randomly (1514) proceed to either branch 1506 or 1518 to determine the frequency and timing of the AF-triggered survey. If true, health monitor 96 will then proceed to determine if the patient has completed either survey in the last 25 days (1518). If true, health monitor 96 may prompt a survey for a random day in the next 11 days from today (1520). If the patient has not completed a survey in the last 25 days, health monitor 96 will then check if the last survey was sent more than 25 days ago (1522). If true, health monitor 96 may prompt a survey for a random day in the next 11 days from today (1520).

If health monitor 96 determines that the last survey was not sent more than 25 days ago, health monitor 96 will then determine if there are no previous instances of either survey completed by the patient (1524). If true, health monitor 96 may prompt a survey for a random day in the next 11 days from today (1520). If false, health monitor 96 does not prompt a survey (1526).

In some examples, external device 12 (e.g., executing health monitor 96) may be configured to send reminders, as well as the initial survey prompts, using any of the notification techniques described above. In some examples, health monitor 96 may be configured to alter the schedule of both survey prompts and reminders. For example, computing system 20 may gather data indicating the completion rate of surveys for both initial prompts and reminders. Based on analysis of complete rates, computing system 20 may update health monitor 96 to operate according to an updated schedule for survey prompts and reminders that has been shown to increase patient compliance. Health monitor 96 may be configure to vary one or more of a frequency, time of day, day, time in the study (e.g., first 3 months), etc. to the schedule of sending survey prompts and reminders.

In another example of the disclosure, external device 12 (e.g., executing health monitor 96) may be configured to aid patient 4 in self-enrollment in a clinical study. Health monitor 96 may be configured to present self-enrollment documents (or provide a link to self-enrollment documents) based on the patient being part of a participating network. In general, based on patient parameters, location within a geofenced area, general proximity to a study site, collaborators, health conditions, physicians, health insurance companies, and/or healthcare network, health monitor 96 may be configured to provide self-enrollment documents to patients where you might be able to gather more information. In other examples, health monitor 96 may be configured to cause external device 12 to read QR codes that provide access to self-enrollment documents. In some examples, external device 12 may be configured to read a QR code that is used to download health monitor 96 and/or another application or website that allows for enrollment in the study.

Typically in clinical trials, study sponsors, like Medtronic, focus on a fixed number of clinical study sites to focus company resources on an often long and complex study initiation process. Due to the resources required to involve each study site, sites are assessed on a number of metrics aimed at de-risking their participation. However, this process is expensive and time consuming, with the study sponsor holding most of the risk.

This disclosure further proposes the development and posting of study site participation materials in a common, website-based access point that would allow interested study sites to take it upon themselves to sign the appropriate documentation that legally allows the site to participate in a study. In some examples, health monitor 96 may provide access or links to such websites. The study sites would then carry the risk for qualifying themselves and enrolling patients without needing to be selected by the sponsor's clinical organization or independently activated by the sponsor's clinical team, reducing costs incurred to the study sponsor and allowing for greater patient inclusion.

Legal documents that modify the terms and conditions related to the collection and study of patient data may be posted on an online study webpage, in addition to an overview of the process an interested study site would need to follow to ensure the study site can participate. The interested study site may obtain an authorized account owner for that clinical site to sign the agreement document and get the study materials, including but not limited to, the study protocol, informed consent, and HIPAA authorization forms through their appropriate oversight committees (e.g., institutional review board). The study site may need to determine their ability to provide data of sufficient quality and how it can be collected. Once this work is done, the study sponsor can then add that study site to its list of participating centers, provide study materials, and allow the site to begin patient enrollment.

Finding eligible patients for our clinical studies is typically a challenging process. In some examples, study sponsors have relied on clinical study sites to recruit patients using a number of methods. Research coordinators often search electronic health records for their institution to locate patients that meet the study inclusion/exclusion criteria. Additionally, physicians may assess patients during clinical visits for their study candidacy.

Rather than relying on the study site alone to identify eligible patients, health monitor 96 may be configured to further gather information for identifying study participants. Based on a patient's involvement in a research study, health monitor 96 may gather demographic, physiological, medical history, and/or device data that could be used to determine eligibility for another clinical study supported by health monitor 96. For example, health monitor 96 may be used in a study related to AF and may and learn that a particular patient also has heart failure. In this case, health monitor 96 may prompt the patient if they would like to participate in in a heart failure study in addition to the AF study.

It may be beneficial to obtain patient consent at the time of study enrollment to be contacted about future clinical studies. Health monitor 96 may retain some patient-related data pertaining to key inclusion/exclusion characteristics of potential future studies. Additionally, when new studies are added to health monitor 96, health monitor 96 may be configured to analyze search fields of patient data that specify inclusion/exclusion criteria for the new study to determine eligibility based on the stored data from prior studies. Example criteria may include: chronic diseases, age, gender, hospitalizations, etc.

In other examples of the disclosure, health monitor 96 and/or computing system 20 may be configured to validate a patient-input device serial number and eligibility criteria during clinical trial enrollment. More specifically, health monitor 96 and/or computing system 20 may be configured to validate whether a patient attempting to remotely enroll in a clinical trial is eligible for enrollment based off of screening criteria, such as medical history and their implanted medical device.

Health monitor 96 may be configured to allow the patient to input the Device Serial Number of IMD 10, and report demographics and medical history needed to determine if the patient has met criteria to enroll in the study. When the patient is deemed eligible, health monitor 96 may be configured to prompt the patient to proceed to create an account and sign consent forms to enroll.

In the example of a Medtronic LINQ device, health monitor 96 may be configured to prompt and collect a 10 digit serial number. Health care monitor queries Medtronic Device Registration API service (Mendix) to determine if serial number exists for a qualifying model. If the serial number provided initially qualifies, health monitor 96 then prompts the patient to provide inputs to various screening questions such as date of birth (DOB), gender, medical history, etc. Health monitor 96 may invoke a second query to the Medtronic device registration API to determine if the device serial number is a likely demographic match based off of the patient-supplied DOB and gender, as well as the device registration DOB and gender. Health monitor 96 may evaluate additional device registration elements input to determine if patient device qualifies for study (e.g., Device Implant Date, Associated Clinic Account, etc.), as well as patient screening questions to determine if the patient qualifies for study.

In other examples, health monitor 96 may be configured to facilitate patient communication with a healthcare provider and/or clinical sponsor. Patient compliance to protocol requirements vary by patient, duration in a trial, and activity being required. There are also time delays to alternate methods of communication, via health portals on EMR database 222, email, and voicemails. By having secure patient communication on health monitor 96, the benefits of direct-timely communication is sourced.

In some examples, a patient is requested to take a health survey every month, since the survey's clinical utility is maximized when taken at fixed intervals. By sending a reminder message on health monitor 96, and also confirming that the activity (e.g., survey) was completed and submitted to a healthcare provider, greater compliance is achieved. Health monitor 96 may provide the patient a summary of the device data for a previous X period of time upon completing an activity. This communication can also be used for ad hoc or pro re nata “PRN” medication request. An example message may be: “Mr. Jones, this is a reminder to take your XXX study drug today and confirm upon completion.”

Health monitor 96 may include a communication tab and also a priority status that would require the action to be acknowledged before moving on. For example, a message that indicates “please acknowledge XX activity has been completed for today,” that is presented before being able to see Diagnostics data on their device. This communication would be one-way so as not to burden a health care provider with messaging coming from the patient. Any communication from the patient would preferably be made directly with a healthcare provider so that time delays on a response are not requirements of health monitor 96. Communication from the Sponsor to the patient could be two-way as defined by reminders for the patient to complete a task and also ask questions on how to complete a task.

Health monitor 96 may be configured to collect information related to activities that are required by the study or healthcare provide to assess compliance. Patient compliance may be tested vs outcomes. Patient communication can also be tested vs outcomes. Such tests may indicate whether a patient that is more engaged in their healthcare show better results. Such tests may further indicate how does communication with a caretaker/family member improves outcomes and/or how healthcare providers accept one-way communication vs testing two-way communication.

In other examples, health monitor 96 may facilitate patient referral to another healthcare provider or caretaker. In one example, healthcare provider #1 sees a patient in clinic for a standard follow-up and diagnoses another condition that he/she does not routinely follow. Healthcare provider #1 can send a referral for that patient through their EMR system, but the patient is not part of communication path.

In accordance with the techniques of this disclosure, health monitor 96 may be configured to include communication capabilities that allow for referrals to other healthcare providers so that the patient is in the communication loop between the two healthcare providers.

All parties would have access to health monitor 96 (e.g., as a healthcare provider access vs patient access). The patient could add physicians in their medical network as parties they would be acceptable seeing for medical care. The healthcare provider would also have updates as to how many patients were referred by whom/to whom and if a subsequent clinic visit was complete.

The techniques performed by external device 12 (e.g., executing health monitor 96) and computing system 20 may increase patient engagement and improving health outcomes. Clinical studies are costly and time-intensive, and keeping participants engaged and compliant during multi-year studies is essential to the success of the study outcomes. For fully remote studies, this issue of engagement and compliance is compounded. Thus, the problem this disclosure seeks to address is how to drive continuous participant engagement and compliance in a remote, mobile app-based, multi-year clinical study directed at patients with an implanted medical device (e.g., a Reveal LINQ and LINQ II device).

There is high demand among patients with an implanted heart device to see their device data, yet patients with the Reveal LINQ and LINQ II devices with bedside monitor or Bluetooth connectivity currently cannot view device metrics without contacting their overseeing physician.

The techniques of this disclosure include leveraging this desire for device data by providing patients who are enrolled in an app-based study with access to curated data from their Reveal LINQ or LINQ II device on the study's mobile app (e.g., health monitor 96). Allowing patients to have access to their data through an app-based direct to patient registry will drive engagement so that the study can reduce data missingness and improve patient compliance.

Eligible patients who have an implanted Reveal LINQ or LINQ II device will confirm their identity during in-app study enrollment by providing their device serial number, which will be verified via CareLink to confirm that they are part of a clinic associated with the study. Once the patient has successfully completed enrollment, they will be presented with an in-app device data acknowledgment screen to ensure they understand what is being presented and confirm that it is not intended to affect their current treatment. After acknowledgement, the patient will be presented with a data view that pulls specific data elements from the CareLink System and presents it within the app on a 24-48 hour delay.

In some examples, health monitor 96 may be configured to collect Healthkit and interaction behavior. As it pertains to analytics in the main data views, health monitor 96 may collect data on page views and time spent per session. For patients who opt to share their Healthkit data, health monitor 96 may be configured to collect the following data:
allergyRecord
conditionRecord
labResultRecord
medicationRecord
procedureRecord
vitalSignRecord
activityMoveMode
biologicalSex
dateOfBirth
distanceWalkingRunning
basalEnergyBurned
activeEnergyBurned
appleExerciseTime
appleStandHour
appleStandtime
height
bodyMass
bodyMassIndex
leanBodyMass
bodyFatPercentage
waistCircumference
heartRate
lowHeartRateEvent
highHeartRateEvent
irregularHeartRhythmEvent
restingHeartRate
heartRateVariabilitySDNN
walkingHeartRateAverage
HKDataTypeIdentifierHeartbeatSeries
HKElectrocardiogramType
oxygenSaturation
bloodPressure
bloodPressureSystolic
bloodPressureDiastolic
respiratoryRate
vO2Max
Nutrition
Symptoms
bloodGlucose
insulinDelivery
peripheralPerfusionIndex
sleepAnalysis
Workouts

For those patients who do not opt to share data, health monitor 96 will not be collecting anything from Healthkit. Patient device data being pulled from CareLink will not be pushed to HealthKit.

In addition, health monitor 96 may include a survey as part of the study to assess the impact of giving patients their heart device data. This is survey referenced as the Patient Experience and Satisfaction questionnaire. Health monitor 96 may deploy this survey every 6 months, starting 6-months post-enrollment.

Questions include but are not limited to:
Do you look at your LINQ device data in this app?
If you do not routinely look at your LINQ device data in the app, why not?
Has seeing your LINQ device data caused you to manage your atrial fibrillation condition differently?
Which LINQ device data presented in this app has provided you with meaningful information?
Do you feel more anxious or less anxious about your atrial fibrillation after seeing your LINQ device data?
After viewing your LINQ device data, do you feel a need to contact your care provider about your atrial fibrillation?
Does viewing your LINQ device data allow you to have more informed conversations about your health with your care provider?
   Leveraging this data collected from the study will result in opportunities to provide more transparency to patients by enabling more programs to show device data to their end consumers.

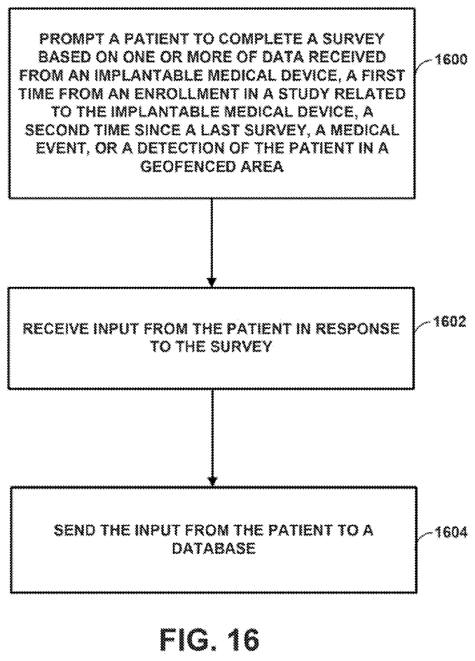

FIG. 16 is a flowchart illustrating an example techniques of the disclosure. The techniques of FIG. 16 may be performed by one or more of external device 12, computing system 20, and/or sensor device 14.

In one example of the disclosure, external device 12 (e.g., executing health monitor 96) may be configured to prompt a patient to complete a survey based on one or more of data received from an implantable medical device, a first time from an enrollment in a study related to the implantable medical device, a second time since a last survey, a medical event, or a detection of the patient in a geofenced area (1600). External device 12 may be further configured to receive input from the patient in response to the survey (1602), and send the input from the patient to a database (1604).

In one example, the implantable medical device is an implantable cardiac monitor. In this example, the data received from the implantable cardiac monitor is data indicative of a clinical event of interest, such an atrial fibrillation event. In one example, to prompt the patient to complete the survey, external device 12 is configured to prompt the patent to complete the survey based on a single atrial fibrillation event lasting longer than a predetermined threshold. In one example, the predetermined threshold is one hour. In another example, to prompt the patient to complete the survey, external device 12 is configured to prompt the patent to complete the survey based on a cumulative daily atrial fibrillation burden being greater than a predetermined threshold. In this example, the predetermined threshold is 5%.

In another example, to prompt the patient to complete the survey, external device 12 is configured to prompt the patient to complete the survey based on two or more of the data received from the implantable medical device, the first time from enrollment in the study related to the implantable medical device, the second time since the last survey, the medical event, or the detection of the patient in the geofenced area.

In another example, to prompt the patient to complete the survey, external device 12 is configured to prompt the patient to complete the survey based on the detection of the patient in the geofenced area for a third time greater than a predetermined threshold. In this example, the geofenced area is proximate to a clinic related to the study.

In another example, to prompt the patient to complete the survey, external device 12 is configured to prompt the patient to complete the survey based on the detection of the patient in the geofenced area for a third time greater than a predetermined threshold if the patient has not completed a previous survey for more than a predetermined period of time. In one example, to prompt the patient to complete the survey, external device 12 is configured to prompt the patient to complete the survey on the same day as the detection of the patient in the geofenced area.

In another example, to prompt the patient to complete the survey, external device 12 is configured to prompt the patient to complete the survey on a random day within X number of days based on at least the second time since the last survey.

In one example, the survey is related to patient symptoms. In another example, the survey is related to healthcare utilization. In another example, the survey is related to quality-of-life. In still another example, the survey is related to medication.

In another example, to prompt the patient to complete the survey, external device 12 is configured to send a push notification to a mobile device of the patient.

In another example, external device 12 is further configured to send the data received from the implantable medical device to the database.

In another example, to prompt the patient to complete the survey, external device 12 is configured to prompt the patient, at regular intervals, to complete the survey based on one or more of the data received from the implantable medical device, the first time from enrollment in the study related to the implantable medical device, the second time since the last survey, the medical event, or the detection of the patient in the geofenced area.

In another example, to prompt the patient to complete the survey, external device 12 is configured to prompt the patient, at a random time within a time interval, to complete the survey based on one or more of the data received from the implantable medical device, the first time from enrollment in the study related to the implantable medical device, the second time since the last survey, the medical event, or the detection of the patient in the geofenced area.

In another example, external device 12 is configured to send a reminder to the patient to complete the survey.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

Examples include:

Example 1. A method for processing patient data, the method comprising:
prompting, by a computing device, a patient to complete a survey based on one or more of data received from an implantable medical device, a first time from an enrollment in a study related to the implantable medical device, a second time since a last survey, a medical event, or a detection of the patient in a geofenced area;
receiving, by the computing device, input from the patient in response to the survey; and
sending, by the computing device, the input from the patient to a database.

Example 2. The method of Example 1, wherein prompting the patient to complete the survey comprises:
prompting the patient to complete the survey on a random day within X number of days based on at least the second time since the last survey.

Example 3. The method of any of Examples 1-2, wherein the survey is related to patient symptoms.

Example 4. The method of any of Examples 1-3, wherein the survey is related to healthcare utilization.

Example 5. The method of any of Examples 1-4, wherein the survey is related to quality-of-life.

Example 6. The method of any of Examples 1-5, wherein the survey is related to medication.

Example 7. The method of any of Examples 1-6, wherein prompting the patient to complete the survey comprises:
sending a push notification to a mobile device of the patient.

Example 8. The method of any of Examples 1-7, further comprising: accessing the survey on the mobile device.

Example 9. The method of any of Examples 1-8, further comprising: sending the data received from the implantable medical device to the database.

Example 10. The method of any of Examples 1-9, wherein prompting the patient to complete the survey comprises:
prompting the patient, at regular intervals, to complete the survey based on one or more of the data received from the implantable medical device, the first time from enrollment in the study related to the implantable medical device, the second time since the last survey, the medical event, or the detection of the patient in the geofenced area.

Example 11. The method of any of Examples 1-10, wherein prompting the patient to complete the survey comprises:
prompting the patient, at a random time within a time interval, to complete the survey based on one or more of the data received from the implantable medical device, the first time from enrollment in the study related to the implantable medical device, the second time since the last survey, the medical event, or the detection of the patient in the geofenced area.

Example 12. The method of any of Examples 1-11, further comprising: sending a reminder to the patient to complete the survey.

Example 13. The method of any combination of techniques of Examples 1-12.

Example 14. A device configured to process patient data, the device comprising:
a memory; and
one or more processors in communication with the memory, the one or more processors configured to perform any combination of techniques of Examples 1-12.

Example 15. A non-transitory computer-readable storage medium storing instructions that, when executed, cause one or more processors of a device configured to process patient data to perform any combination of techniques of Examples 1-12.

Example 16. Any combination of techniques described in this disclosure.

1. A device configured to process patient data, the device comprising:
a memory;
a user interface; and
one or more processors in communication with the memory and the user interface, the one or more processors configured to:
prompt, via the user interface, a patient to complete a survey based on one or more of data received from an implantable medical device or a detection of the patient in a geofenced area;
receive, via the user interface, input from the patient in response to the survey; and
cause the input from the patient to be sent to a database.

2. The device of claim 1, wherein the implantable medical device is an implantable cardiac monitor.

3. The device of claim 2, wherein the data received from the implantable cardiac monitor is data indicative of a clinical event of interest.

4. The device of claim 3, wherein the clinical event of interest is an atrial fibrillation event.

5. The device of claim 4, wherein the one or more processors configured to prompt, via the user interface, the patient to complete the survey comprises:
prompt, via the user interface, the patent to complete the survey based on a single atrial fibrillation event lasting longer than a predetermined threshold.

6. The device of claim 5, wherein the predetermined threshold is one hour.

7. The device of claim 4, wherein the one or more processors configured to prompt, via the user interface, the patient to complete the survey comprises:
prompt, via the user interface, the patent to complete the survey based on a cumulative daily atrial fibrillation burden being greater than a predetermined threshold.

8. The device of claim 7, wherein the predetermined threshold is 5%.

9. The device of claim 1, wherein the one or more processors configured to prompt, via the user interface, the patient to complete the survey comprises:
prompt the patient to complete the survey based on both the data received from the implantable medical device and the detection of the patient in the geofenced area.

10. The device of claim 1, wherein the one or more processors configured to prompt, via the user interface, the patient to complete the survey comprises:
prompt, via the user interface, the patient to complete the survey based on the detection of the patient in the geofenced area for a third time greater than a predetermined threshold.

11. The device of claim 10, wherein the geofenced area is proximate to a clinic related to the study.

12. The device of claim 1, wherein the one or more processors configured to prompt, via the user interface, the patient to complete the survey comprises:
prompt, via the user interface, the patient to complete the survey based on the detection of the patient in the geofenced area for a third time greater than a predetermined threshold if the patient has not completed a previous survey for more than a predetermined period of time.

13. The device of claim 12, wherein the one or more processors configured to prompt, via the user interface, the patient to complete the survey comprises:
prompt, via the user interface, the patient to complete the survey on the same day as the detection of the patient in the geofenced area.

14. A non-transitory computer-readable storage medium storing instructions that, when executed, cause one or more processors of a device configured to process patient data to perform:
prompting, by the device, a patient to complete a survey based on one or more of data received from an implantable medical device, a first time from an enrollment in a study related to the implantable medical device, a second time since a last survey, a medical event, or a detection of the patient in a geofenced area;
receiving, by device, input from the patient in response to the survey; and
sending, by the device, the input from the patient to a database.

15. A method for processing patient data, the method comprising:
prompting, by a computing device, a patient to complete a survey based on one or more of data received from an implantable medical device, a first time from an enrollment in a study related to the implantable medical device, a second time since a last survey, a medical event, or a detection of the patient in a geofenced area;
receiving, by the computing device, input from the patient in response to the survey; and
sending, by the computing device, the input from the patient to a database.

16. The method of claim 15, wherein the implantable medical device is an implantable cardiac monitor.

17. The method of claim 16, wherein the data received from the implantable cardiac monitor is data indicative of a clinical event of interest.

18. The method of claim 17, wherein the clinical event of interest is an atrial fibrillation event.

19. The method of claim 18, wherein prompting the patient to complete the survey comprises:
prompting the patent to complete the survey based on a single atrial fibrillation event lasting longer than a predetermined threshold.

20. The method of claim 19, wherein the predetermined threshold is one hour.
